(19) United States
(12) Patent Application Publication
(10) Pub. No.: US 2026/0337160 A1
(43) Pub. Date: Oct. 29, 2026

(54) PREPARATION AND PURIFICATION METHOD FOR INTERMEDIATE OF METHYL AURISTATIN E COMPOUND

(71) Applicant: REMEGEN CO., LTD., Yantai (CN)

(72) Inventors: Marie M. ZHU, Yantai (CN); Haitao LI, Yantai (CN); Xin LIANG, Yantai (CN); Xinli LI, Yantai (CN); Xiaoqing SUN, Yantai (CN); Peng SUN, Yantai (CN); Kai XIAO, Yantai (CN); Zemin JU, Yantai (CN); Mengmeng JIANG, Yantai (CN)

(73) Assignee: REMEGEN CO., LTD., Yantai (CN)

(21) Appl. No.: 19/480,356

(22) PCT Filed: May 28, 2024
(86) PCT No.: PCT/CN2024/095719

§ 371 Date: Oct. 30, 2025

(30) Foreign Application Priority Data
Jun. 2, 2023 (CN) 202310657645.1

(51) Int. Cl. C07D 207/09

(52) U.S. Cl. CPC C07D 207/09

(57) ABSTRACT

A preparation and purification method for an intermediate of a methyl auristatin E compound. The preparation route of the compound is as follows: the preparation and purification method comprises the following steps: A, dissolving compound 1, compound 2, a polypeptide condensing agent and an organic alkali in an organic solvent 1, and reacting same, so as to obtain a solution 1 after the reaction is finished; B, adding an organic solvent 2 and water to the solution 1, and carrying out extraction; and C, collecting an organic phase, and concentrating same, so as to obtain a compound as shown in formula (I), wherein R is an amino protecting group. The product purification method is simple, and a target product with a high purity and a high yield can be obtained simply by means of one-step extraction and concentration, such that not only production steps, the consumption of production and production inputs can be effectively reduced, but also the yield of the product can be effectively improved; and the requirements for equipment and environment are not high, good stability is achieved among multiple batches, the color of the final product is better, and the method is more suitable for scale-up production.

### FIELD

The present disclosure relates to the field of methods for synthesizing compounds, in particular to a method for producing and purifying monomethyl auristatin E (MMAE) compound.

### BACKGROUND

An antibody-drug conjugate (ADC) is an anti-tumor drug, comprising three parts: an antibody moiety, a linker moiety, and a toxin (drug) moiety, wherein the antibody moiety and the toxin moiety are conjugated via the linker. The mechanism of action of ADCs involves delivering the drug to target cells (such as tumor cells) utilizing the targeting ability of the antibody, where the drug is released to kill the tumor cells. At present, although a large quantity of natural or chemically synthesized cytotoxins are known, only a very small fraction of them can be applied in ADCs. It is mainly because toxins used as ADC payloads must possess a combination of characteristics, such as an extremely high cytotoxicity, an intracellular target, and a small molecular structure. Among them, auristatin compounds (such as MMAE and MMAF) are cytotoxic molecules that have been successfully used in the development of multiple ADC drugs.

MMAE (also referred to as monomethyl auristatin E), a chemically synthesized auristatin derivative, can effectively inhibit mitosis by inhibiting tubulin polymerization. At present, the market price of MMAE is very high, partly because currently known synthetic routes of MMAE are relatively complicated, involving multiple steps and resulting in low yields. Additionally, the purification processes of the prior art are complex, involving complicated steps and requiring a large amount of reagents, most of which cannot be recovered or require an additional recovery step, and resulting in higher production costs. At present, a common route for producing MMAE is as follows:

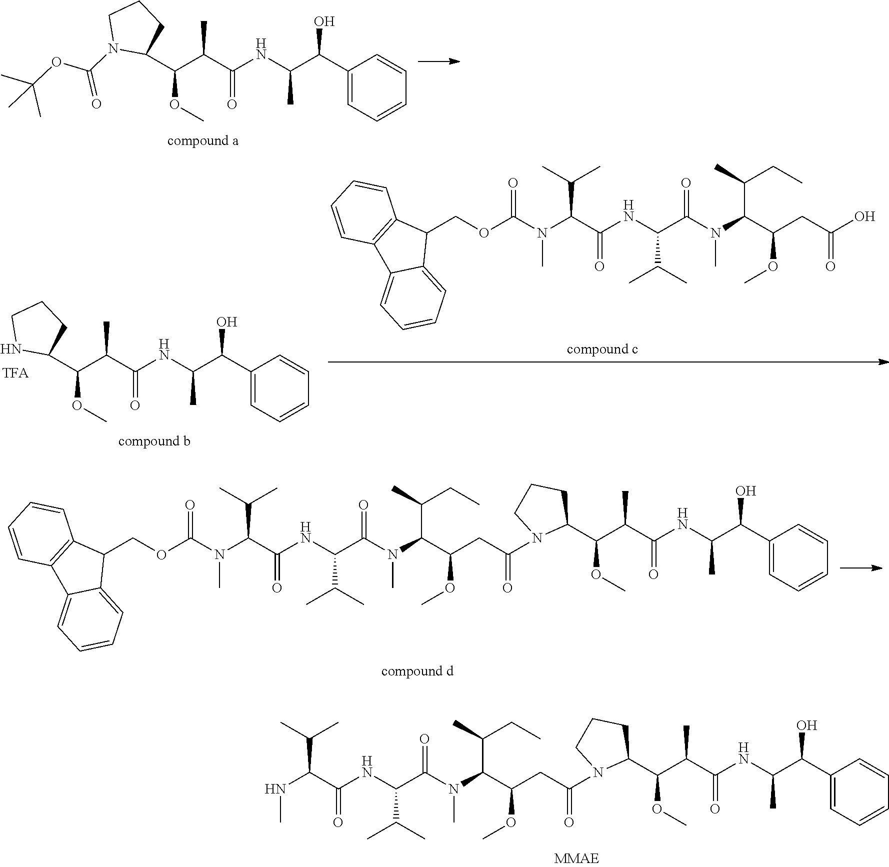

In the route above, compound d is a main precursor compound. A preparation strategy of compound d is disclosed in Chinese patent publication No. CN105143199A: dissolving compound b and compound c in DCM under an inert atmosphere, cooling the solution to 0° C., adding DEPC and DIEA dropwise, stirring the reaction mixture at 0° C. for 2 h and then at ambient temperature overnight, diluting the reaction mixture with 100 mL of water, extracting the reaction mixture with 50 mL of DCM three times, combining the organic phases, washing the organic phase successively with KHSO₄, NaHCO₃, and NaCl once each, drying the organic phase over sodium sulfate, filtering the mixture, and concentrating the filtrate under a reduced pressure, to obtain compound d.

The method for preparing compound d disclosed in aforementioned publication has a relatively low yield of only 39% (see paragraph [0328] of the specification of CN105143199A). In addition, the process for purifying compound d disclosed in aforementioned publication is relatively complicated, requiring washing successively with KHSO₄, NaHCO₃, and NaCl after extraction in the purification process. This complicated process results in significant inconvenience for large-scale production and post-treatment, elevates production cost, and is not suitable for industrial application.

### SUMMARY

In view of the foregoing problems, the present disclosure provides a method for producing and purifying an intermediate of monomethyl auristatin E compound, which achieves a high yield, involves a simple purification process, and produces a product with high purity and stable quality.

Specifically, the present disclosure provides a method for producing and purifying a compound of formula (I) (namely an intermediate of monomethyl auristatin E of the present disclosure):

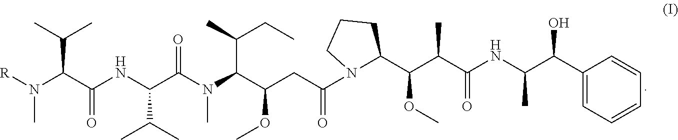

The compound has a production route of:

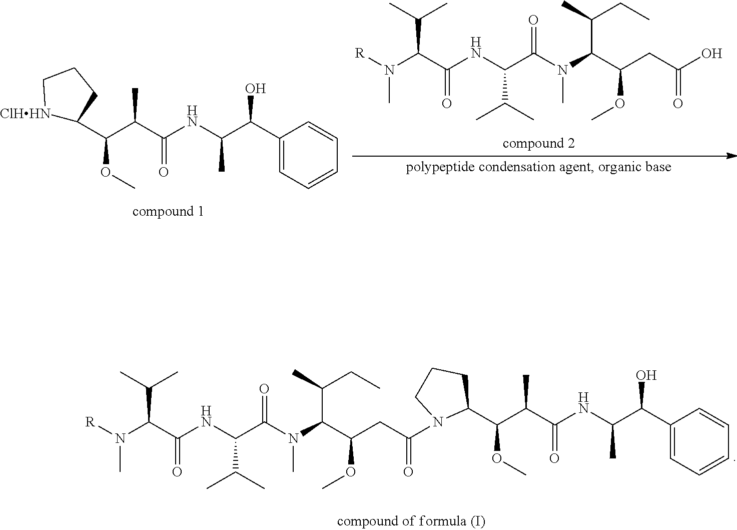

The method for producing and purifying the compound of formula (I) comprises the following steps:
A. dissolving compound 1, compound 2, a polypeptide condensation condensation agent, and an organic base in an organic solvent 1, and reacting;
B. after the reaction in step A is complete, adding an organic solvent 2 and water to the resulting solution, and extracting;
C. collecting and concentrating organic phase, to obtain the compound of formula (I).

Wherein R is an amino protecting group. The amino protecting group of the present disclosure is a group introduced into a molecule for protecting the amino group. In some embodiments, R is selected from the group consisting of an Fmoc protecting group, a Boc protecting group, and a Cbz protecting group as described below:

| | |
|---|---|
| Fmoc protecting group (9-fluorenylmethyloxycarbonyl) | 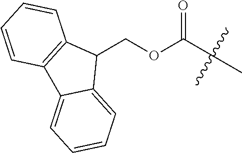 [SMILES: *C(=O)OCC1c2ccccc2-c2ccccc21] |
| Boc protecting group (tert-butoxycarbonyl) | 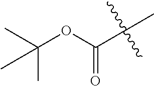 [SMILES: *C(=O)OC(C)(C)C] |
| Cbz protecting group (benzyloxycarbonyl) | 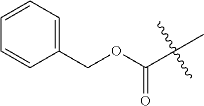 [SMILES: *C(=O)OCc1ccccc1] |

Organic solvent 1 is any one or more selected from the group consisting of N,N-dimethylformamide (CAS No: 127-19-5), N-methylpyrrolidone (CAS No: 872-50-4), methanol, ethanol, isopropanol, acetonitrile, acetone, tetrahydrofuran, acetonitrile, and 1,4-dioxane.

Organic solvent 2 is any one or more selected from the group consisting of n-heptane, petroleum ether, n-hexane, cyclohexane, n-pentane, methylcyclohexane, and methyl tert-butyl ether.

The polypeptide condensation agent is a reaction adjuvant for the condensation reaction of carboxylic acids with amino groups. In some embodiments, the polypeptide condensation agent is any one or more selected from the group consisting of HATU (CAS No: 148893-10-1, N,N,N′,N′-tetramethyl-O-(7-azabenzotriazol-1-yl) uronium hexafluorophosphate), HBTU (CAS No: 94790-37-1, benzotriazol-N,N,N′,N′-tetramethyluronium hexafluorophosphate), and TBTU (CAS No: 125700-67-6, O-benzotriazol-N,N,N′,N′-tetramethyluronium tetrafluoroborate).

The organic base is an organic compound containing an amino group in its molecule. In some embodiments, the organic base is any one or more selected from the group consisting of DIPEA (CAS No: 7087-68-5, N,N-diisopropylethylamine), triethylamine (CAS No: 121-44-8), 4-methylmorpholine (CAS No: 109-02-4), and pyridine (CAS No: 110-86-1).

Step A is performed under an inert gas atmosphere; preferably, the inert gas is nitrogen, argon, or helium gas.

In some embodiments, organic solvent 1 is N,N-dimethylformamide; in some other embodiments, organic solvent 1 is N-methylpyrrolidone; in some other embodiments, organic solvent 1 is methanol; in some other embodiments, organic solvent 1 is ethanol; in some other embodiments, organic solvent 1 is isopropanol; in some other embodiments, organic solvent 1 is acetonitrile; in some other embodiments, organic solvent 1 is acetone; in some other embodiments, organic solvent 1 is tetrahydrofuran; in some other embodiments, organic solvent 1 is acetonitrile; in some other embodiments, organic solvent 1 is 1,4-dioxane; in some other embodiments, organic solvent 1 is any one or more of N,N-dimethylformamide, N-methylpyrrolidone, methanol, ethanol, isopropanol, acetonitrile, acetone, tetrahydrofuran, acetonitrile, and 1,4-dioxane, such as (including but not limited to) a mixed solution of N,N-dimethylformamide and N-methylpyrrolidone (with a volume ratio of 1:1, 1:2, 1:3, and the like), a mixed solution of methanol and ethanol (with a volume ratio of 1:1, 1:2, 1:3, and the like), a mixed solution of isopropanol and acetonitrile (with a volume ratio of 1:1, 1:2, 1:3, and the like), a mixed solution of acetone and tetrahydrofuran (with a volume ratio of 1:1, 1:2, 1:3, and the like), a mixed solution of acetonitrile and 1,4-dioxane (with a volume ratio of 1:1, 1:2, 1:3, and the like), and the like.

In some embodiments, organic solvent 2 is n-heptane; in some other embodiments, organic solvent 2 is petroleum ether; in some other embodiments, organic solvent 2 is n-hexane; in some other embodiments, organic solvent 2 is cyclohexane; in some other embodiments, organic solvent 2 is n-pentane; in some other embodiments, organic solvent 2 is methylcyclohexane; in some other embodiments, organic solvent 2 is methyl tert-butyl ether.

In some embodiments, organic solvent 1 and organic solvent 2 may be selected independently.

In some embodiments, the polypeptide condensation agent is HATU; in some other embodiments, the polypeptide condensation agent is HBTU; in some other embodiments, the polypeptide condensation agent is TBTU; in some other embodiments, the polypeptide condensation agent is a mixture of HATU and HBTU, HATU and TBTU, or HBTU and TBTU, in any ratio; in some other embodiments, the polypeptide condensation agent is a mixture of HATU, HBTU and TBTU in any ratio.

In some embodiments, the organic base is DIPEA; in some other embodiments, the organic base is triethylamine; in some other embodiments, the organic base is 4-methylmorpholine; in some other embodiments, the organic base is pyridine; in some other embodiments, the organic base is a mixture of DIPEA and triethylamine, DIPEA and 4-methylmorpholine, DIPEA and pyridine, triethylamine and 4-methylmorpholine, triethylamine and pyridine, or 4-methylmorpholine and pyridine, in any ratio.

Furthermore, compound 2 has a structure selected from:

| | |
|---|---|
| 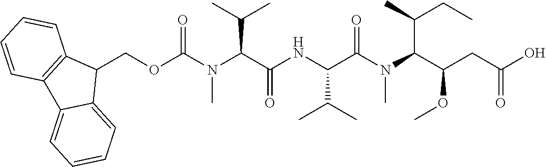 [SMILES: CC[C@H](C)[C@@H]([C@@H](CC(=O)O)OC)N(C)C(=O)[C@@H](NC(=O)[C@H](C(C)C)N(C)C(=O)OCC1c2ccccc2-c2ccccc21)C(C)C] | (2-1) |
| 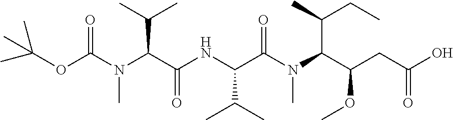 [SMILES: CC[C@H](C)[C@@H]([C@@H](CC(=O)O)OC)N(C)C(=O)[C@@H](NC(=O)[C@H](C(C)C)N(C)C(=O)OC(C)(C)C)C(C)C] | (2-2) |
| 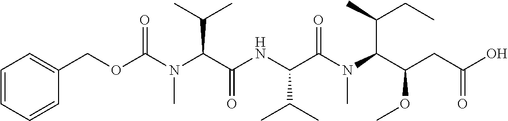 [SMILES: CC[C@H](C)[C@@H]([C@@H](CC(=O)O)OC)N(C)C(=O)[C@@H](NC(=O)[C@H](C(C)C)N(C)C(=O)OCc1ccccc1)C(C)C] | (2-3) |

Furthermore, the compound of formula (I) has a structure selected from:

| | |
|---|---|
| 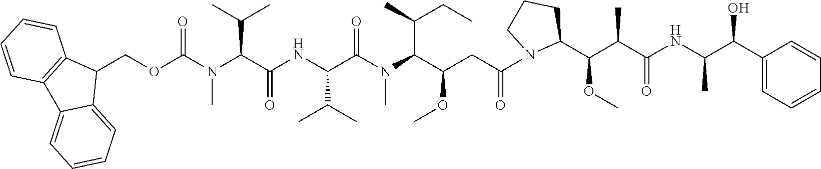 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCC1c2ccccc2-c2ccccc21)C(C)C] | (I-1) |
| 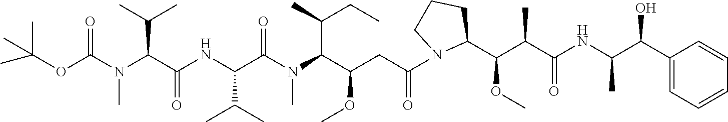 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OC(C)(C)C)C(C)C] | (I-2) |
| 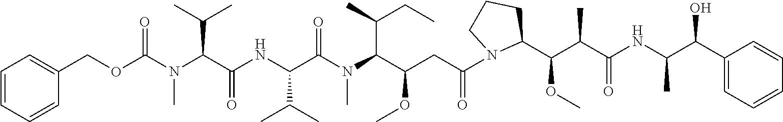 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccccc1)C(C)C] | (I-3) |

In one embodiment, the compound of formula (I-1) has a production route of:

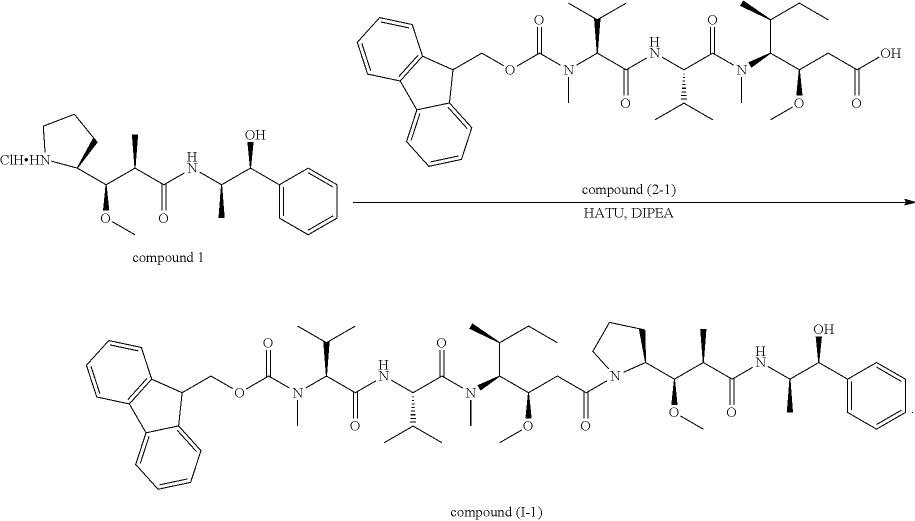

In one embodiment, the compound of formula (I-2) has a production route of:

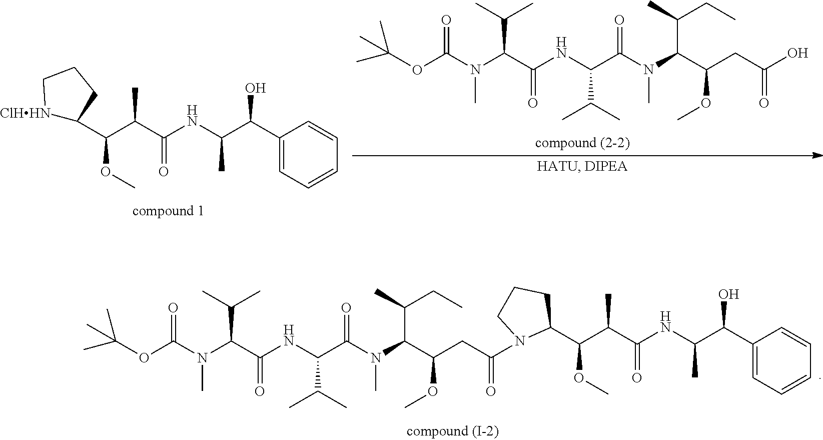

In one embodiment, the compound of formula (I-3) has a production route of:

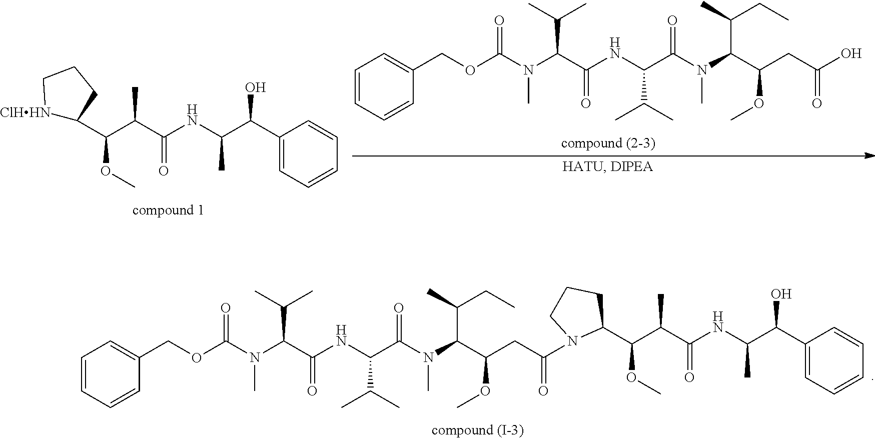

Furthermore, in step A, compound 1 and compound 2 have a mole ratio of 1:1 to 1:2; in one embodiment, in step A, compound 1 and compound 2 have a mole ratio of 1:2; in one embodiment, in step A, compound 1 and compound 2 have a mole ratio of 1:1.95; in one embodiment, in step A, compound 1 and compound 2 have a mole ratio of 1:1.9; in one embodiment, in step A, compound 1 and compound 2 have a mole ratio of 1:1.85; in one embodiment, in step A, compound 1 and compound 2 have a mole ratio of 1:1.8; in one embodiment, in step A, compound 1 and compound 2 have a mole ratio of 1:1.75; in one embodiment, in step A, compound 1 and compound 2 have a mole ratio of 1:1.7; in one embodiment, in step A, compound 1 and compound 2 have a mole ratio of 1:1.65; in one embodiment, in step A, compound 1 and compound 2 have a mole ratio of 1:1.6; in one embodiment, in step A, compound 1 and compound 2 have a mole ratio of 1:1.55; in one embodiment, in step A, compound 1 and compound 2 have a mole ratio of 1:1.5; in one embodiment, in step A, compound 1 and compound 2 have a mole ratio of 1:1.45; in one embodiment, in step A, compound 1 and compound 2 have a mole ratio of 1:1.4; in one embodiment, in step A, compound 1 and compound 2 have a mole ratio of 1:1.35; in one embodiment, in step A, compound 1 and compound 2 have a mole ratio of 1:1.3; in one embodiment, in step A, compound 1 and compound 2 have a mole ratio of 1:1.25; in one embodiment, in step A, compound 1 and compound 2 have a mole ratio of 1:1.2; in one embodiment, in step A, compound 1 and compound 2 have a mole ratio of 1:1.15; in one embodiment, in step A, compound 1 and compound 2 have a mole ratio of 1:1.1; in one embodiment, in step A, compound 1 and compound 2 have a mole ratio of 1:1.05; in one embodiment, in step A, compound 1 and compound 2 have a mole ratio of 1:1; in one embodiment, in step A, compound 1 and compound 2 have a mole ratio of 1:0.95; in one embodiment, in step A, compound 1 and compound 2 have a mole ratio of 1:0.9; in one embodiment, in step A, compound 1 and compound 2 have a mole ratio of 1:0.85; in one embodiment, in step A, compound 1 and compound 2 have a mole ratio of 1:0.8; in one embodiment, in step A, compound 1 and compound 2 have a mole ratio of 1:0.75; in one embodiment, in step A, compound 1 and compound 2 have a mole ratio of 1:0.7.

Furthermore, in step A, compound 1 and the polypeptide condensation agent have a mole ratio of 1:1 to 1:2; in one embodiment, in step A, compound 1 and the polypeptide condensation agent have a mole ratio of 1:2; in one embodiment, in step A, compound 1 and the polypeptide condensation agent have a mole ratio of 1:1.95; in one embodiment, in step A, compound 1 and the polypeptide condensation agent have a mole ratio of 1:1.9; in one embodiment, in step A, compound 1 and the polypeptide condensation agent have a mole ratio of 1:1.85; in one embodiment, in step A, compound 1 and the polypeptide condensation agent have a mole ratio of 1:1.8; in one embodiment, in step A, compound 1 and the polypeptide condensation agent have a mole ratio of 1:1.75; in one embodiment, in step A, compound 1 and the polypeptide condensation agent have a mole ratio of 1:1.7; in one embodiment, in step A, compound 1 and the polypeptide condensation agent have a mole ratio of 1:1.65; in one embodiment, in step A, compound 1 and the polypeptide condensation agent have a mole ratio of 1:1.6; in one embodiment, in step A, compound 1 and the polypeptide condensation agent have a mole ratio of 1:1.55; in one embodiment, in step A, compound 1 and the polypeptide condensation agent have a mole ratio of 1:1.5; in one embodiment, in step A, compound 1 and the polypeptide condensation agent have a mole ratio of 1:1.45; in one embodiment, in step A, compound 1 and the polypeptide condensation agent have a mole ratio of 1:1.4; in one embodiment, in step A, compound 1 and the polypeptide condensation agent have a mole ratio of 1:1.35; in one embodiment, in step A, compound 1 and the polypeptide condensation agent have a mole ratio of 1:1.3; in one embodiment, in step A, compound 1 and the polypeptide condensation agent have a mole ratio of 1:1.25; in one embodiment, in step A, compound 1 and the polypeptide condensation agent have a mole ratio of 1:1.2; in one embodiment, in step A, compound 1 and the polypeptide condensation agent have a mole ratio of 1:1.15; in one embodiment, in step A, compound 1 and the polypeptide condensation agent have a mole ratio of 1:1.1; in one embodiment, in step A, compound 1 and the polypeptide condensation agent have a mole ratio of 1:1.05; in one embodiment, in step A, compound 1 and the polypeptide condensation agent have a mole ratio of 1:1; in one embodiment, in step A, compound 1 and the polypeptide condensation agent have a mole ratio of 1:0.95; in one embodiment, in step A, compound 1 and the polypeptide condensation agent have a mole ratio of 1:0.9; in one embodiment, in step A, compound 1 and the polypeptide condensation agent have a mole ratio of 1:0.85; in one embodiment, in step A, compound 1 and the polypeptide condensation agent have a mole ratio of 1:0.8; in one embodiment, in step A, compound 1 and the polypeptide condensation agent have a mole ratio of 1:0.75; in one embodiment, in step A, compound 1 and the polypeptide condensation agent have a mole ratio of 1:0.7.

Furthermore, in step A, compound 1 and the organic base have a mole ratio of 1:2 to 1:5; in one embodiment, in step A, compound 1 and the organic base have a mole ratio of 1:5; in one embodiment, in step A, compound 1 and the organic base have a mole ratio of 1:4.9; in one embodiment, in step A, compound 1 and the organic base have a mole ratio of 1:4.8; in one embodiment, in step A, compound 1 and the organic base have a mole ratio of 1:4.7; in one embodiment, in step A, compound 1 and the organic base have a mole ratio of 1:4.6; in one embodiment, in step A, compound 1 and the organic base have a mole ratio of 1:4.5; in one embodiment, in step A, compound 1 and the organic base have a mole ratio of 1:4.4; in one embodiment, in step A, compound 1 and the organic base have a mole ratio of 1:4.3; in one embodiment, in step A, compound 1 and the organic base have a mole ratio of 1:4.2; in one embodiment, in step A, compound 1 and the organic base have a mole ratio of 1:4.1; in one embodiment, in step A, compound 1 and the organic base have a mole ratio of 1:4; in one embodiment, in step A, compound 1 and the organic base have a mole ratio of 1:3.9; in one embodiment, in step A, compound 1 and the organic base have a mole ratio of 1:3.8; in one embodiment, in step A, compound 1 and the organic base have a mole ratio of 1:3.7; in one embodiment, in step A, compound 1 and the organic base have a mole ratio of 1:3.6; in one embodiment, in step A, compound 1 and the organic base have a mole ratio of 1:3.5; in one embodiment, in step A, compound 1 and the organic base have a mole ratio of 1:3.4; in one embodiment, in step A, compound 1 and the organic base have a mole ratio of 1:3.3; in one embodiment, in step A, compound 1 and the organic base have a mole ratio of 1:3.2; in one embodiment, in step A, compound 1 and the organic base have a mole ratio of 1:3.1; in one embodiment, in step A, compound 1 and the organic base have a mole ratio of 1:3; in one embodiment, in step A, compound 1 and the organic base have a mole ratio of 1:2.9; in one embodiment, in step A, compound 1 and the organic base have a mole ratio of 1:2.8; in one embodiment, in step A, compound 1 and the organic base have a mole ratio of 1:2.7; in one embodiment, in step A, compound 1 and the organic base have a mole ratio of 1:2.6; in one embodiment, in step A, compound 1 and the organic base have a mole ratio of 1:2.5; in one embodiment, in step A, compound 1 and the organic base have a mole ratio of 1:2.4; in one embodiment, in step A, compound 1 and the organic base have a mole ratio of 1:2.3; in one embodiment, in step A, compound 1 and the organic base have a mole ratio of 1:2.2; in one embodiment, in step A, compound 1 and the organic base have a mole ratio of 1:2.1; in one embodiment, in step A, compound 1 and the organic base have a mole ratio of 1:2.

Furthermore, in step A, compound 1 and organic solvent 1 have a weight-to-volume ratio (g/mL) of 1:5 to 1:15; in one embodiment, in step A, compound 1 and organic solvent 1 have a weight-to-volume ratio (g/mL) of 1:15; in one embodiment, in step A, compound 1 and organic solvent 1 have a weight-to-volume ratio (g/mL) of 1:14; in one embodiment, in step A, compound 1 and organic solvent 1 have a weight-to-volume ratio (g/mL) of 1:13; in one embodiment, in step A, compound 1 and organic solvent 1 have a weight-to-volume ratio (g/mL) of 1:12; in one embodiment, in step A, compound 1 and organic solvent 1 have a weight-to-volume ratio (g/mL) of 1:11; in one embodiment, in step A, compound 1 and organic solvent 1 have a weight-to-volume ratio (g/mL) of 1:10; in one embodiment, in step A, compound 1 and organic solvent 1 have a weight-to-volume ratio (g/mL) of 1:9; in one embodiment, in step A, compound 1 and organic solvent 1 have a weight-to-volume ratio (g/mL) of 1:8; in one embodiment, in step A, compound 1 and organic solvent 1 have a weight-to-volume ratio (g/mL) of 1:7; in one embodiment, in step A, compound 1 and organic solvent 1 have a weight-to-volume ratio (g/mL) of 1:6; in one embodiment, in step A, compound 1 and organic solvent 1 have a weight-to-volume ratio (g/mL) of 1:5.

Furthermore, step A is performed at a temperature of 0 to 30° C.

It should be understood that, in some embodiments, step A is performed at a temperature that may fluctuate within a certain range (such as ±5° C., ±10° C., ±15° C.). In one embodiment, step A is performed at a temperature of 0° C.±5° C.; in one embodiment, step A is performed at a temperature of 1° C.±5° C.; in one embodiment, step A is performed at a temperature of 2° C.±5° C.; in one embodiment, step A is performed at a temperature of 3° C.±5° C.; in one embodiment, step A is performed at a temperature of 4° C.±5° C.; in one embodiment, step A is performed at a temperature of 5° C.±5° C.; in one embodiment, step A is performed at a temperature of 6° C.±5° C.; in one embodiment, step A is performed at a temperature of 7° C.±5° C.; in one embodiment, step A is performed at a temperature of 8° C.±5° C.; in one embodiment, step A is performed at a temperature of 9° C.±5° C.; in one embodiment, step A is performed at a temperature of 10° C.±5° C.; in one embodiment, step A is performed at a temperature of 11° C.±5° C.; in one embodiment, step A is performed at a temperature of 12° C.±5° C.; in one embodiment, step A is performed at a temperature of 13° C.±5° C.; in one embodiment, step A is performed at a temperature of 14° C.±5° C.; in one embodiment, step A is performed at a temperature of 15° C.±5° C.; in one embodiment, step A is performed at a temperature of 16° C.±5° C.; in one embodiment, step A is performed at a temperature of 17° C.±5° C.; in one embodiment, step A is performed at a temperature of 18° C.±5° C.; in one embodiment, step A is performed at a temperature of 19° C.±5° C.; in one embodiment, step A is performed at a temperature of 20° C.±5° C.; in one embodiment, step A is performed at a temperature of 21° C.±5° C.; in one embodiment, step A is performed at a temperature of 22° C.±5° C.; in one embodiment, step A is performed at a temperature of 23° C.±5° C.; in one embodiment, step A is performed at a temperature of 24° C.±5° C.; in one embodiment, step A is performed at a temperature of 25° C.±5° C.; in one embodiment, step A is performed at a temperature of 26° C.±5° C.; in one embodiment, step A is performed at a temperature of 27° C.±5° C.; in one embodiment, step A is performed at a temperature of 28° C.±5° C.; in one embodiment, step A is performed at a temperature of 29° C.±5° C.; in one embodiment, step A is performed at a temperature of 30° C.±5° C.

In one embodiment, step A is performed at a temperature of 0 to 5° C., 10 to 15° C., 20 to 30° C., 0 to 15° C., or 0 to 10° C.

Furthermore, compound 1 in step A and organic solvent 2 in step B have a weight-to-volume ratio (g/mL) of 1:5 to 1:15. In one embodiment, compound 1 in step A and organic solvent 2 in step B have a weight-to-volume ratio (g/mL) of 1:5; in one embodiment, compound 1 in step A and organic solvent 2 in step B have a weight-to-volume ratio (g/mL) of 1:6; in one embodiment, compound 1 in step A and organic solvent 2 in step B have a weight-to-volume ratio (g/mL) of 1:7; in one embodiment, compound 1 in step A and organic solvent 2 in step B have a weight-to-volume ratio (g/mL) of 1:8; in one embodiment, compound 1 in step A and organic solvent 2 in step B have a weight-to-volume ratio (g/mL) of 1:9; in one embodiment, compound 1 in step A and organic solvent 2 in step B have a weight-to-volume ratio (g/mL) of 1:10; in one embodiment, compound 1 in step A and organic solvent 2 in step B have a weight-to-volume ratio (g/mL) of 1:11; in one embodiment, compound 1 in step A and organic solvent 2 in step B have a weight-to-volume ratio (g/mL) of 1:12; in one embodiment, compound 1 in step A and organic solvent 2 in step B have a weight-to-volume ratio (g/mL) of 1:13; in one embodiment, compound 1 in step A and organic solvent 2 in step B have a weight-to-volume ratio (g/mL) of 1:14; in one embodiment, compound 1 in step A and organic solvent 2 in step B have a weight-to-volume ratio (g/mL) of 1:15.

Furthermore, compound 1 in step A and the water in step B have a weight-to-volume ratio (g/mL) of 1:5 to 1:15. In one embodiment, compound 1 in step A and the water in step B have a weight-to-volume ratio (g/mL) of 1:5; in one embodiment, compound 1 in step A and the water in step B have a weight-to-volume ratio (g/mL) of 1:6; in one embodiment, compound 1 in step A and the water in step B have a weight-to-volume ratio (g/mL) of 1:7; in one embodiment, compound 1 in step A and the water in step B have a weight-to-volume ratio (g/mL) of 1:8; in one embodiment, compound 1 in step A and the water in step B have a weight-to-volume ratio (g/mL) of 1:9; in one embodiment, compound 1 in step A and the water in step B have a weight-to-volume ratio (g/mL) of 1:10; in one embodiment, compound 1 in step A and the water in step B have a weight-to-volume ratio (g/mL) of 1:11; in one embodiment, compound 1 in step A and the water in step B have a weight-to-volume ratio (g/mL) of 1:12; in one embodiment, compound 1 in step A and the water in step B have a weight-to-volume ratio (g/mL) of 1:13; in one embodiment, compound 1 in step A and the water in step B have a weight-to-volume ratio (g/mL) of 1:14; in one embodiment, compound 1 in step A and the water in step B have a weight-to-volume ratio (g/mL) of 1:15.

In one embodiment, the water in step B is iced water.

In one embodiment, the concentrating in step C is performed under a reduced pressure.

The present disclosure also provides a use of any one of the aforementioned methods in production for MMAE.

The present disclosure also provides a use of any one of the aforementioned methods in production for an intermediate of MMAE.

The present disclosure also provides a use of the method for producing and/or purifying the aforementioned intermediate of monomethyl auristatin E compound (namely compound of formula (I)) in production for an antibody-drug conjugate containing MMAE as a toxin.

The present disclosure also provides a use of the method for producing and/or purifying the aforementioned intermediate of monomethyl auristatin E compound (namely compound of formula (I)) in production for an intermediate of an antibody-drug conjugate containing MMAE as a toxin.

The method for producing and purifying an intermediate of monomethyl auristatin E compound (namely compound of formula (I)) provided in the present disclosure features a simple product purification process, in which target product with a high purity (>99%) can be obtained only by a step of extracting and concentrating. In addition, the method provided in the present disclosure has a high yield (up to 95% to 105%), which is beneficial for simplifying production process, reducing production consumption and input, and increasing production yield. Therefore, target product with a high yield and a high purity can be obtained while reducing energy consumption. The production efficiency is significantly improved, the consumption of the reagents is effectively reduced, and the production cost is reduced. Besides, due to the simple process, the low requirements for equipment and environment, the high process stability, and the good batch-to-batch stability of the method provided by the present disclosure, the final product has a better color, a more stable quality, and thereby the method is more suitable for large-scale production.

### BRIEF DESCRIPTION OF DRAWINGS

FIG. 1 is a chromatogram of compound (I-1) prepared in Example 1;

FIG. 2 is a chromatogram of compound (I-2) prepared in Example 2;

FIG. 3 is a chromatogram of compound (I-3) prepared in Example 3;

FIG. 4 is a chromatogram of compound (I-1) prepared in Example 4;

FIG. 5 is a chromatogram of compound (I-1) prepared in Example 5;

FIG. 6 is a chromatogram of compound (I-1) prepared in Comparative Example 1.

### DETAILED DESCRIPTION

The embodiments of the present disclosure is further described in detail below in a non-limiting manner in combination with specific embodiments. It should be noted that the following embodiments are only for illustrating the embodiments of the present disclosure, with the purpose of enabling those in the art to understand the content of the present disclosure and practice the same, but are not intended to limit the protection scope of the present disclosure. All equivalent changes or modifications made according to the spirit of the present disclosure should be encompassed within the protection scope of the present disclosure.

### Example 1: Preparation of Compound (I-1)

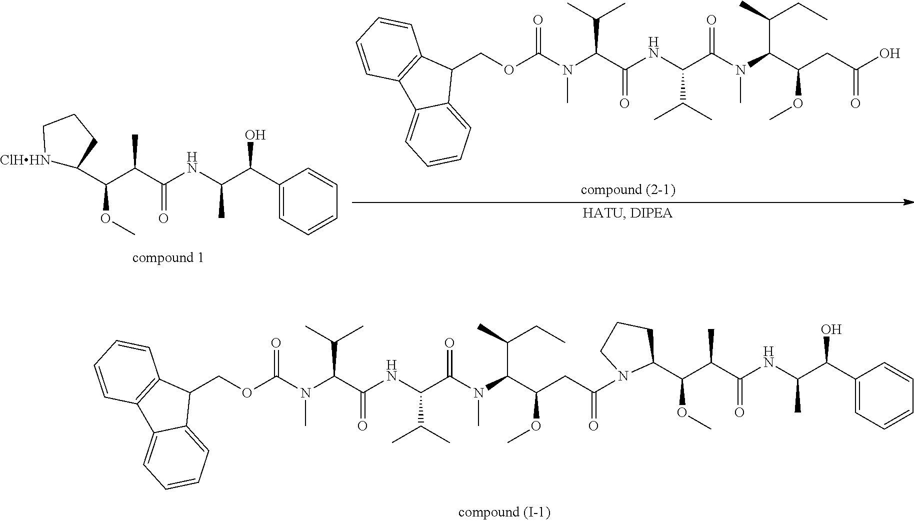

To a three-necked bottle, compound 1 (namely (2R,3R)—N-((1S,2R)-1-hydroxy-1-phenylpropan-2-yl)-3-methoxy-2-methyl-3-((S)-pyrrolidin-2-yl) propanamide hydrochloride, 1.0 g, 2.8 mmol), compound 2-1 (namely (5S, 8S, 11S, 12R)-11-((S)-sec-butyl)-1-(9H-fluoren-9-yl)-5,8-diisopropyl-12-methoxy-4,10-dimethyl-3,6,9-tri oxo-2-oxa-4,7,10-triazatetradecan-14-oic acid, 1.88 g, 2.95 mmol), HATU (namely 2-(7-azabenzotriazol)-N,N,N′,N′-tetramethyluronium hexafluorophosphate, 1.28 g, 3.4 mmol), DIPEA (namely N,N-diisopropylethylamine, 1.09 g, 8.4 mmol), and DMF (namely N,N-dimethylformamide, 9 mL, 9 vol) were added and reacted at a temperature of 0 to 5° C. under nitrogen protection.

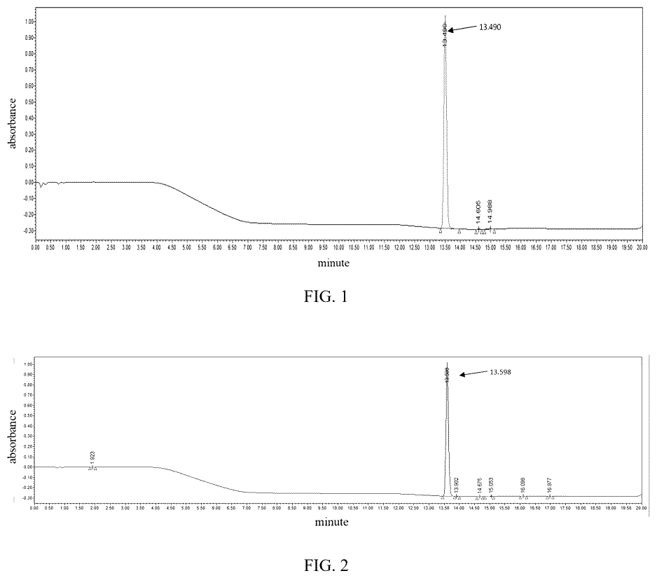

After the reaction was complete, the reaction solution was transferred to a separatory funnel, methyl tert-butyl ether (10 mL) and iced water (10 mL) were added successively for extraction, and the organic phase was separated. The organic phase was concentrated under reduced pressure to a foam in a 100 mL single-neck round-bottom flask, and dried under vacuum for no less than 1 h until the weight no longer changed, 2.59 g of compound I-1 (namely (9H-fluoren-9-yl)methyl((S)-1-(((S)-1-(3R,4S,5S)-1-(S)-2-((1R,2R)-3-((1S,2R)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl(methyl)amino)-3-methyl-1-oxobutan-2-yl)amino)-3-methyl-1-oxobutan-2-yl)(methyl)carbamate) was obtained with a yield of 98.2%. The product had a white color. Analysis of a sample showed a purity of 99.67% with the largest single impurity content being 0.20%. The chromatogram was shown in FIG. 1.

### Example 2: Preparation of Compound (I-2)

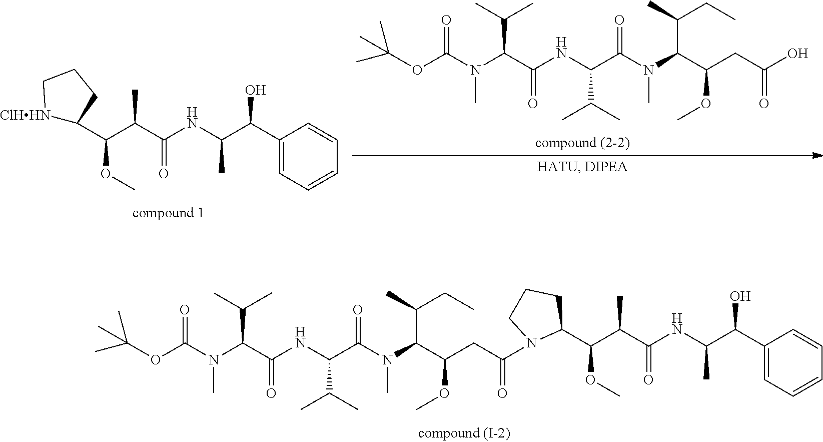

To a three-necked bottle, compound 1 (namely (2R,3R)—N-((1S,2R)-1-hydroxy-1-phenylpropan-2-yl)-3-methoxy-2-methyl-3-((S)-pyrrolidin-2-yl)propanamide hydrochloride, 20.0 g, 56 mmol), compound 2-2 (namely (6S, 9S, 12S, 13R)-12-((S)-sec-butyl)-6,9-diisopropyl-13-methoxy-2,2,5,11-tetramethyl-4,7,10-trioxo-3-oxa-5,8,11-triazapentadec-15-enoic acid, 34.68 g, 67.2 mmol), HATU (namely 2-(7-azabenzotriazol)-N,N,N′,N′-tetramethyluronium hexafluorophosphate, 23.44 g, 61.6 mmol), DIPEA (namely N,N-diisopropylethylamine, 28.97 g, 224 mmol), and N-methylpyrrolidone (280 mL, 14 vol) were added and reacted at a temperature of 10 to 15° C. under nitrogen protection.

After the reaction was complete, the reaction solution was transferred to a separatory funnel, petroleum ether (280 mL) and iced water (280 mL) were added successively for extraction, and the organic phase was separated. The organic phase was concentrated under reduced pressure to a foam in a 1000 mL single-neck round-bottom flask, and dried under vacuum for no less than 1 h until the weight no longer changed, 45.75 g of compound I-2 (namely (tert-butyl(S)-1-(S)-1-(3R,4S,4S,5S,5S)-1-(S)-2-((1R,2R,2R)-3-(1S,2Rs)-1-hydroxy-1-phenylpropan-2-phenylpropan-2-phenylpropan-2-phenylpropan-1-methoxymethylmethoxy-1-methoxymethylmethyl-1-methoxyethylethyl-1-methoxymethoxy-3-methoxy-5-methyl-1-oxohept an-4-methylmethylmethylmethyl-methylmethylmethyl-3-methylmethyl-3-methoxymethyl-3-methyl-3-methyl-1-methyl-1-oxobutan-2-yl)(methyl)carbamate) was obtained with a yield of 99.8%. The product had an off-white color. Analysis of a sample showed a purity of 99.44% with the largest single impurity content being 0.26%. The chromatogram was shown in FIG. 2.

### Example 3: Preparation of Compound (I-3)

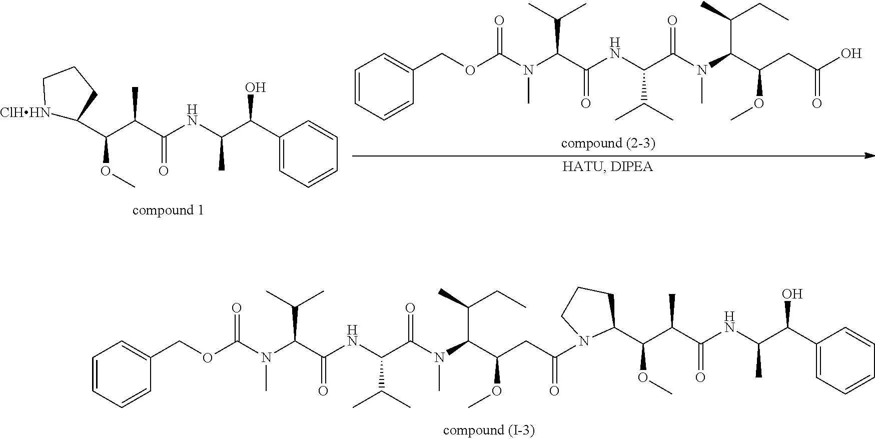

To a three-necked bottle, compound 1 (namely (2R,3R)—N-((1S,2R)-1-hydroxy-1-phenylpropan-2-yl)-3-methoxy-2-methyl-3-((S)-pyrrolidin-2-yl)propanamide hydrochloride, 5.0 g, 14 mmol), compound 2-3 (namely (5S, 8S, 11S, 12R)-11-((S)-sec-butyl)-5,8-diisopropyl-12-methoxy-4,10-dimethyl-3,6,9-trioxo-1-phenyl-2-oxa-4,7,10-triazatetradecan-14-oic acid, 11.55 g, 21 mmol), HATU (namely 2-(7-azabenzotriazol)-N,N,N′,N′-tetramethyluronium hexafluorophosphate, 5.33 g, 14 mmol), DIPEA (namely N,N-diisopropylethylamine, 9.05 g, 70 mmol), and methanol (25 mL, 5 vol) were added and reacted at a temperature of 20 to 30° C. under nitrogen protection.

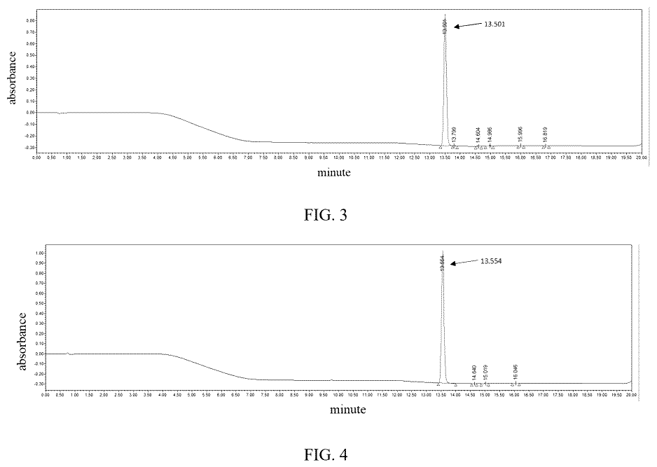

After the reaction was complete, the reaction solution was transferred to a separatory funnel, n-heptane (40 mL) and iced water (40 mL) were added successively for extraction, and the organic phase was separated. The organic phase was concentrated under reduced pressure to a foam in a 250 mL single-neck round-bottom flask, and dried under vacuum for no less than 1 h until the weight no longer changed, 11.59 g of compound I-3 (namely (S)-1-((S)-1-[(3R,4S,5S)-1-(S)-2-(1R,2R)-3-(((1S,2R)1-hydroxy-1-phenylpropan-2-yl)amino)-1-methoxy-2-methyl-3-oxopropyl)pyrrolidin-1-yl)-3-methoxy-5-methyl-1-oxoheptan-4-yl(methyl)amino)-3-methyl-1-oxobutan-2-yl)-3-methyl-1-oxobutan-2-yl-(methyl)carbamate)(methyl)carbamate) was obtained with a yield of 97.1%. The product had an off-white color. Analysis of a sample showed a purity of 99.46% with the largest single impurity content being 0.27%. The chromatogram was shown in FIG. 3.

### Example 4: Preparation of Compound (I-1)

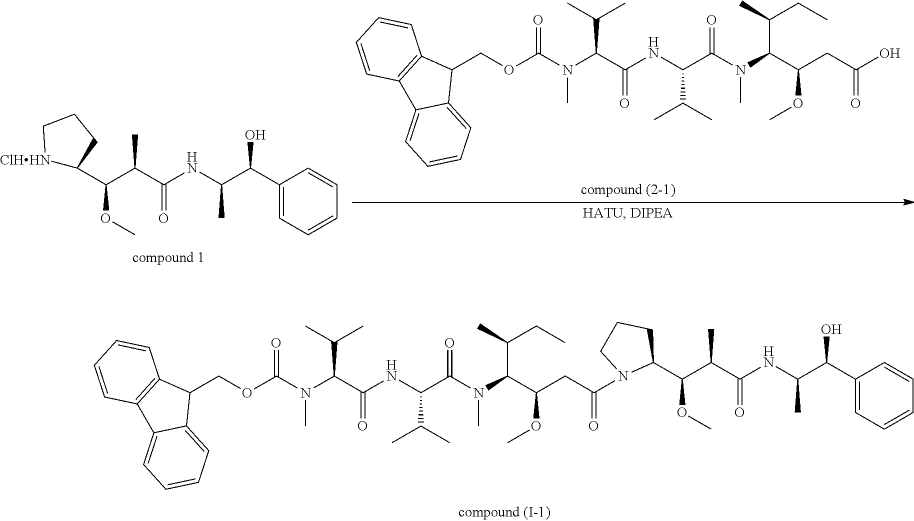

To a three-necked bottle, compound 1 (namely (2R,3R)—N-((1S,2R)-1-hydroxy-1-phenylpropan-2-yl)-3-methoxy-2-methyl-3-((S)-pyrrolidin-2-yl) propanamide hydrochloride, 2.0 g, 5.6 mmol), compound 2-1 (namely (5S, 8S, 11S, 12R)-11-((S)-sec-butyl)-1-(9H-fluoren-9-yl)-5,8-diisopropyl-12-methoxy-4,10-dimethyl-3,6,9-trioxo-2-oxa-4,7,10-triazatetradecan-14-oic acid, 3.57 g, 5.6 mmol), HATU (namely 2-(7-azabenzotriazol)-N,N,N′,N′-tetramethyluronium hexafluorophosphate, 2.13 g, 5.6 mmol), DIPEA (namely N,N-diisopropylethylamine, 1.45 g, 11.2 mmol), and DMF (namely N,N-dimethylformamide, 10 mL) were added and reacted at a temperature of 0 to 15° C. under nitrogen protection.

After the reaction was complete, the reaction solution was transferred to a separatory funnel, methyl tert-butyl ether (10 mL) and iced water (10 mL) were added successively for extraction, and the organic phase was separated. The organic phase was concentrated under reduced pressure to a foam in a 100 mL single-neck round-bottom flask, and dried under vacuum for no less than 1 h until the weight no longer changed, 5.03 g of compound I-1 (namely (9H-fluoren-9-yl)methyl((S)-1-(((S)-1-(3R,4S,5S)-1-(S)-2-((1R,2R)-3-((1S,2R)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl(methyl)amino)-3-methyl-1-oxobutan-2-yl)amino)-3-methyl-1-oxobutan-2-yl)(methyl)carbamate) was obtained with a yield of 95.6%. The product had a white color. Analysis of a sample showed a purity of 99.61% with the largest single impurity content being 0.23%. The chromatogram was shown in FIG. 4.

### Example 5: Preparation of Compound (I-1)

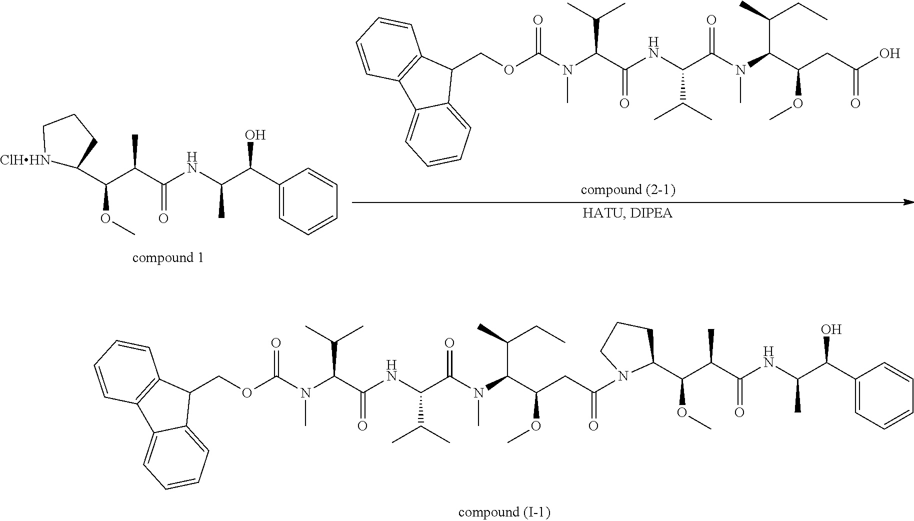

To a three-necked bottle, compound 1 (namely (2R,3R)—N-((1S,2R)-1-hydroxy-1l-phenylpropan-2-yl)-3-methoxy-2-methyl-3-((S)-pyrrolidin-2-yl) propanamide hydrochloride, 2.0 g, 5.6 mmol), compound 2-1 (namely (5S, 8S, 11S, 12R)-11-((S)-sec-butyl)-1-(9H-fluoren-9-yl)-5,8-diisopropyl-12-methoxy-4,10-dimethyl-3,6,9-tri oxo-2-oxa-4,7,10-triazatetradecan-14-oic acid, 7.14 g, 11.2 mmol), HATU (namely 2-(7-azabenzotriazol)-N,N,N′,N′-tetramethyluronium hexafluorophosphate, 4.26 g, 11.2 mmol), DIPEA (namely N,N-diisopropylethylamine, 2.9 g, 22.4 mmol), and DMF (namely N,N-dimethylformamide, 30 mL) were added and reacted at a temperature of 0 to 10° C. under nitrogen protection.

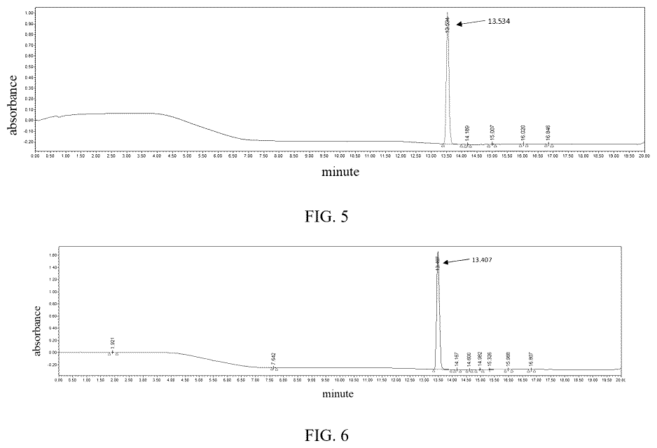

After the reaction was complete, the reaction solution was transferred to a separatory funnel, methyl tert-butyl ether (30 mL) and iced water (30 mL) were added successively for extraction, and the organic phase was separated. The organic phase was concentrated under reduced pressure to a foam in a 100 mL single-neck round-bottom flask, and dried under vacuum for no less than 1 h until the weight no longer changed, 5.24 g of compound I-1 (namely (9H-fluoren-9-yl)methyl((S)-1-(((S)-1-(3R,4S,5S)-1-(S)-2-((1R,2R)-3-((1S,2R)-1-hydroxy-1-phenylpropan-2-yl)amino)-1-methoxy-2-methyl-3-oxopropyl)pyrrolidin-1-yl)-3-meth oxy-5-methyl-1-oxoheptan-4-yl(methyl)amino)-3-methyl-1-oxobutan-2-yl)amino)-3-methyl-1-oxobutan-2-yl)(methyl)carbamate) was obtained with a yield of 99.6%. The product had an off-white color. Analysis of a sample showed a purity of 99.53% with the largest single impurity content being 0.32%. The chromatogram was shown in FIG. 5.

### Comparative Example 1

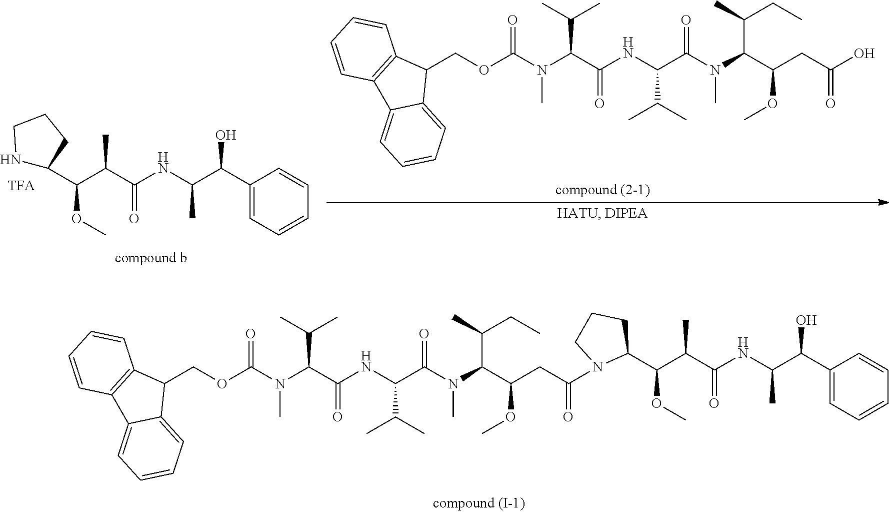 [SMILES: CO[C@@H]([C@@H]1CCCN1)[C@@H](C)C(=O)N[C@H](C)[C@@H](O)c1ccccc1 ; CC[C@H](C)[C@@H]([C@@H](CC(=O)O)OC)N(C)C(=O)[C@@H](NC(=O)[C@H](C(C)C)N(C)C(=O)OCC1c2ccccc2-c2ccccc21)C(C)C ; CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCC1c2ccccc2-c2ccccc21)C(C)C]

To a three-necked bottle, compound b (namely (2R,3R)—N-((1S,2R)-1-hydroxy-1-phenylpropan-2-yl)-3-methoxy-2-methyl-3-((S)-pyrrolidin-2-yl) propanamide trifluoroacetate, 1.0 g, 2.8 mmol), compound 2-1 (namely (5S, 8S, 11S, 12R)-11-((S)-sec-butyl)-1-(9H-fluoren-9-yl)-5,8-diisopropyl-12-methoxy-4,10-dimethyl-3,6,9-tri oxo-2-oxa-4,7,10-triazatetradecan-14-oic acid, 1.60 g, 2.94 mmol), DEPC (namely diethylpyrocarbonate, 0.47 g, 3.4 mmol), DIPEA (namely N,N-diisopropylethylamine, 1.24 g, 11.2 mmol), and dichloromethane (10 mL, 10 vol) were added and reacted at a temperature of 0 to 5° C. under argon protection.

After the reaction was complete, the reaction solution was transferred to a separatory funnel, iced water (15 mL) and dichloromethane (15 mL) were added successively for extraction, and the organic phase was separated. The aqueous phase was then extracted twice more with dichloromethane (15 mL). The organic phase was combined, washed successively with aqueous KHSO₄ solution (15 mL), aqueous NaHCO₃ solution (15 mL), and aqueous NaCl solution (15 mL) once each, dried over anhydrous sodium sulfate (1 g) and filtered. The filtrate was concentrated under reduced pressure to a foam in a 100 mL single-neck round-bottom flask, and dried under vacuum for no less than 1 h until the weight no longer changed, 1.02 g of compound I-1 (namely (9H-fluoren-9-yl)methyl((S)-1-(((S)-1-(3R,4S,5S)-1-(S)-2-((1R,2R)-3-((1S,2R)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl(methyl)amino)-3-methyl-1-oxobutan-2-yl)amino)-3-methyl-1-oxobutan-2-yl)(methyl)carbamate) was obtained with a yield of 45.2%. The product had an off-white color. Analysis of a sample showed a purity of 97.69% with the largest single impurity content being 0.34%. The chromatogram was shown in FIG. 6.

TABLE 1
Comparison of the yields and purities of the products
of different examples and comparative example

| Example | Yield | Purity |
|---|---|---|
| Example 1 | 98.2% | 99.67% |
| Example 2 | 99.8% | 99.44% |
| Example 3 | 97.1% | 99.46% |
| Example 4 | 95.6% | 99.61% |
| Example 5 | 99.6% | 99.53% |
| Comparative Example 1 | 45.2% | 97.69% |

As shown by table 1, the process for producing and purifying an intermediate of monomethyl auristatin E compound provided in the present disclosure had a very high yield of over 95% (even 99.8%). In addition, the product obtained via the process provided in the present disclosure had a high purity of over 99%. While the production and purification process in the prior art had a yield of only 45.2% with a relatively low purity of only 97%, which cannot meet the requirements of further pharmaceutical production. Therefore, the present disclosure has a significant advantage over the prior art.

Furthermore, compared to the prior art in aspects of reaction process and raw materials of reaction/production/purification, the production and purification process of the present disclosure is simpler, involves fewer raw materials, thereby can save a large amount of starting materials and reduce equipment usage, leading to a significant reduction in production costs. Besides, the process provided in the present disclosure is simple, does not require special equipment or environmental conditions, and shows good batch-to-batch product stability. Therefore, the method disclosed by the present disclosure is suitable for industrial application.

The present disclosure has been described through the various specific examples. Various modifications or variations can be made within the scope of the present disclosure, and the various features mentioned throughout this description can be combined with each other without departing from the spirit and scope of the present disclosure. Such modifications and variations are all within the scope of the present disclosure.

1. A method for producing and purifying a compound of formula (I): 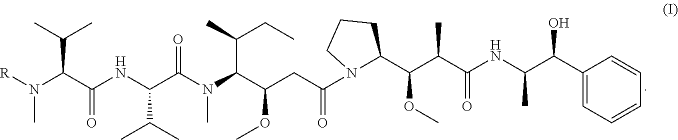
wherein, the compound has a production route of: 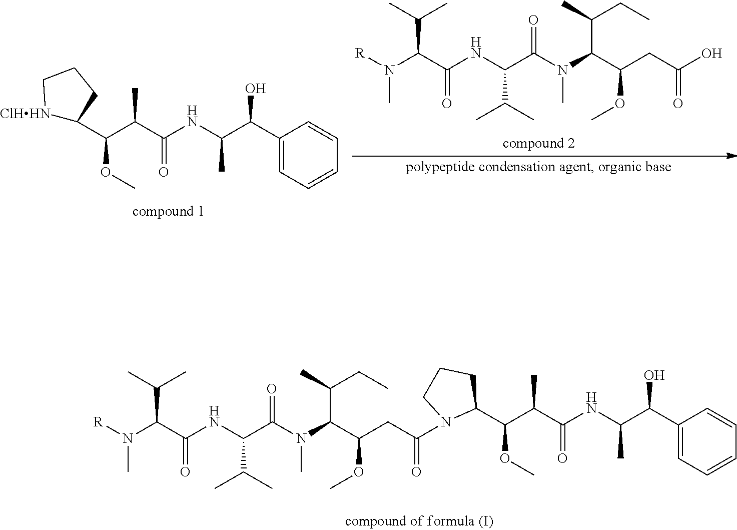
the method for producing and purifying the compound of formula (I) comprises steps of:
A. dissolving compound 1, compound 2, a polypeptide condensation agent, and an organic base in organic solvent 1, and obtaining solution 1 after reaction;
B. adding organic solvent 2 and water to solution 1 and extracting;
C. collecting and concentrating organic phase, to obtain the compound of formula (I);
wherein R is an amino protecting group;
organic solvent 1 is any one or more selected from the group consisting of N,N-dimethylformamide, N-methylpyrrolidone, methanol, ethanol, isopropanol, acetonitrile, acetone, tetrahydrofuran, acetonitrile, and 1,4-dioxane;
organic solvent 2 is any one or more selected from the group consisting of n-heptane, petroleum ether, n-hexane, cyclohexane, n-pentane, methylcyclohexane, and methyl tert-butyl ether;
the polypeptide condensation agent is any one or more selected from the group consisting of HATU, HBTU, and TBTU; and
the organic base is any one or more selected from the group consisting of DIPEA, triethylamine, 4-methylmorpholine, and pyridine.

2. The producing and purifying method according to claim 1, wherein R is selected from the group consisting of an Fmoc protecting group, a Boc protecting group, and a Cbz protecting group, and compound 2 has a structure selected from the following structures:

| | |
|---|---|
| 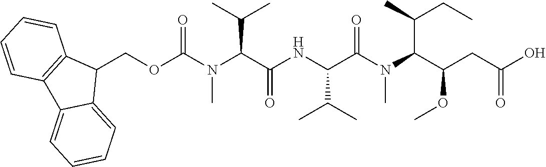 [SMILES: CC[C@H](C)[C@@H]([C@@H](CC(=O)O)OC)N(C)C(=O)[C@@H](NC(=O)[C@H](C(C)C)N(C)C(=O)OCC1c2ccccc2-c2ccccc21)C(C)C] | (2-1) |
| 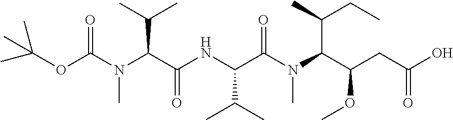 [SMILES: CC[C@H](C)[C@@H]([C@@H](CC(=O)O)OC)N(C)C(=O)[C@@H](NC(=O)[C@H](C(C)C)N(C)C(=O)OC(C)(C)C)C(C)C] | (2-2) |
| 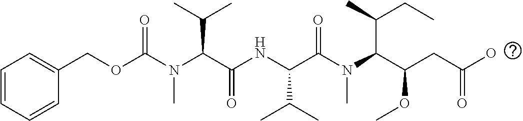 [SMILES: CC[C@H](C)[C@@H]([C@@H](CC(=O)O)OC)N(C)C(=O)[C@@H](NC(=O)[C@H](C(C)C)N(C)C(=O)OCc1ccccc1)C(C)C] [symbol] | (2-3) |

[symbol]

indicates data missing or illegible when filed

3. The producing and purifying method according to claim 2, wherein the compound of formula (I) has a structure selected from:

| | |
|---|---|
| 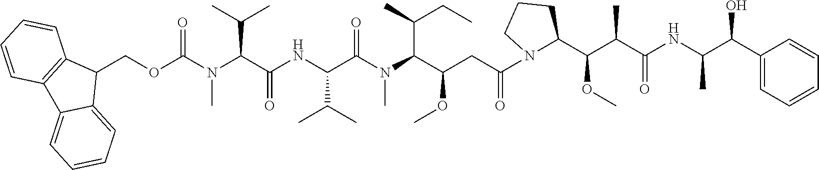 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCC1c2ccccc2-c2ccccc21)C(C)C] | (I-1) |
| 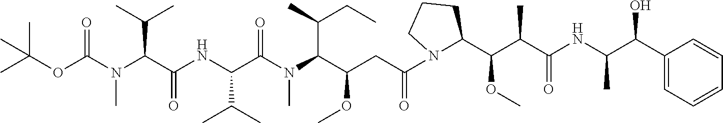 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OC(C)(C)C)C(C)C] | (I-2) |
| 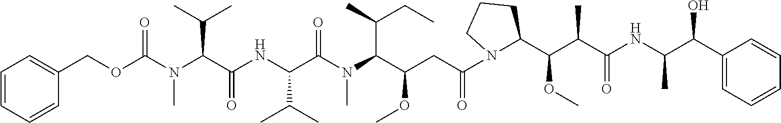 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccccc1)C(C)C] | (I-3) |

4. The producing and purifying method according to claim 1, wherein step A is performed under an inert gas atmosphere; preferably, the inert gas is nitrogen, argon, or helium gas.

5. The producing and purifying method according to claim 1, wherein in step A, compound 1 and compound 2 have a mole ratio of 1:1 to 1:2; preferably, in step A, compound 1 and compound 2 have a mole ratio of 1:1 to 1:1.5; preferably, in step A, compound 1 and compound 2 have a mole ratio of 1:1 to 1:1.2; preferably, in step A, compound 1 and compound 2 have a mole ratio of 1:1 to 1:1.1; preferably, in step A, compound 1 and compound 2 have a mole ratio of 1:1 to 1:1.05; preferably, in step A, compound 1 and compound 2 have a mole ratio of 1:1.05.

6. The producing and purifying method according to claim 1, wherein in step A, compound 1 and the polypeptide condensation agent have a mole ratio of 1:1 to 1:2; preferably, in step A, compound 1 and the polypeptide condensation agent have a mole ratio of 1:1 to 1:1.5; preferably, in step A, compound 1 and the polypeptide condensation agent have a mole ratio of 1:1 to 1:1.4; preferably, in step A, compound 1 and the polypeptide condensation agent have a mole ratio of 1:1 to 1:1.3; preferably, in step A, compound 1 and the polypeptide condensation agent have a mole ratio of 1:1 to 1:1.2; preferably, in step A, compound 1 and the polypeptide condensation agent have a mole ratio of 1:1.2.

7. The producing and purifying method according to claim 1, wherein in step A, compound 1 and the organic base have a mole ratio of 1:2 to 1:5; preferably, in step A, compound 1 and the organic base have a mole ratio of 1:2 to 1:3.5; preferably, in step A, compound 1 and the organic base have a mole ratio of 1:2 to 1:3.4; preferably, in step A, compound 1 and the organic base have a mole ratio of 1:2 to 1:3.3; preferably, in step A, compound 1 and the organic base have a mole ratio of 1:2 to 1:3.2; preferably, in step A, compound 1 and the organic base have a mole ratio of 1:2 to 1:3.1; preferably, in step A, compound 1 and the organic base have a mole ratio of 1:2 to 1:3; preferably, in step A, compound 1 and the organic base have a mole ratio of 1:3.

8. The producing and purifying method according to claim 1, wherein in step A, compound 1 and organic solvent 1 have a weight-to-volume ratio (g/mL) of 1:5 to 1:15; preferably, in step A, compound 1 and organic solvent 1 have a weight-to-volume ratio (g/mL) of 1:5 to 1:14; preferably, in step A, compound 1 and organic solvent 1 have a weight-to-volume ratio (g/mL) of 1:5 to 1:13; preferably, in step A, compound 1 and organic solvent 1 have a weight-to-volume ratio (g/mL) of 1:5 to 1:12; preferably, in step A, compound 1 and organic solvent 1 have a weight-to-volume ratio (g/mL) of 1:5 to 1:11; preferably, in step A, compound 1 and organic solvent 1 have a weight-to-volume ratio (g/mL) of 1:5 to 1:10; preferably, in step A, compound 1 and organic solvent 1 have a weight-to-volume ratio (g/mL) of 1:5 to 1:9; preferably, in step A, compound 1 and organic solvent 1 have a weight-to-volume ratio (g/mL) of 1:9.

9. The producing and purifying method according to claim 1, wherein step A is performed at a temperature of 0 to 30° C.

10. The producing and purifying method according to claim 1, wherein compound 1 in step A and organic solvent 2 in step B have a weight-to-volume ratio (g/mL) of 1:5 to 1:15; preferably, compound 1 in step A and organic solvent 2 in step B have a weight-to-volume ratio (g/mL) of 1:5 to 1:14; preferably, compound 1 in step A and organic solvent 2 in step B have a weight-to-volume ratio (g/mL) of 1:5 to 1:13; preferably, compound 1 in step A and organic solvent 2 in step B have a weight-to-volume ratio (g/mL) of 1:5 to 1:12; preferably, compound 1 in step A and organic solvent 2 in step B have a weight-to-volume ratio (g/mL) of 1:5 to 1:11; preferably, compound 1 in step A and organic solvent 2 in step B have a weight-to-volume ratio (g/mL) of 1:5 to 1:10; preferably, compound 1 in step A and organic solvent 2 in step B have a weight-to-volume ratio (g/mL) of 1:10.

11. The producing and purifying method according to claim 1, wherein compound 1 in step A and the water in step B have a weight-to-volume ratio (g/mL) of 1:5 to 1:15; preferably, compound 1 in step A and the water in step B have a weight-to-volume ratio (g/mL) of 1:5 to 1:14; preferably, compound 1 in step A and the water in step B have a weight-to-volume ratio (g/mL) of 1:5 to 1:13; preferably, compound 1 in step A and the water in step B have a weight-to-volume ratio (g/mL) of 1:5 to 1:12; preferably, compound 1 in step A and the water in step B have a weight-to-volume ratio (g/mL) of 1:5 to 1:11; preferably, compound 1 in step A and the water in step B have a weight-to-volume ratio (g/mL) of 1:5 to 1:10; preferably, compound 1 in step A and the water in step B have a weight-to-volume ratio (g/mL) of 1:10; further preferably, the water in step B is iced water.

12. The producing and purifying method according to claim 1, wherein the concentrating in step C is performed under a reduced pressure.

13. A method for production of an intermediate of MMAE, comprising using the method according to claim 1.

14. A method for production of MMAE, comprising using the method according to claim 1.
